(19) United States
(12) Patent Application Publication
(10) Pub. No.: US 2026/0133175 A1
(43) Pub. Date: May 14, 2026

(54) CORE FLOODING SYSTEM AND METHOD OF ACID DIVERSION STIMULATION

(71) Applicant: KING FAHD UNIVERSITY OF PETROLEUM AND MINERALS, Dhahran (SA)

(72) Inventors: Ahmed Zarzor Hussien YASERI, Dhahran (SA); Abdulameer Mohsin Kadhim ALMALICHY, Miskolc (HU); Murtada Saleh Hassan AL-JAWAD, Dhahran (SA); Xianmin ZHOU, Dhahran (SA)

(73) Assignee: KING FAHD UNIVERSITY OF PETROLEUM AND MINERALS, Dhahran (SA)

(21) Appl. No.: 18/946,725

(22) Filed: Nov. 13, 2024

(30) Foreign Application Priority Data
Nov. 11, 2024 (SA) 1020246327

(51) Int. Cl. G01N 33/24; C09K 8/72; G01N 23/046; G01N 23/06

(52) U.S. Cl. CPC G01N 33/24; C09K 8/72; G01N 23/046; G01N 23/06; G01N 2223/616

(57) ABSTRACT

A core flooding system includes a core holder that includes an outer housing, and an inner housing that is positioned inside the outer housing and includes a first end, a sidewall and a second end. The core holder includes a first discharge port positioned in the first end, a second discharge port positioned in the second end, and injection ports arranged along the sidewall. An acid accumulator is fluidly connected to the injection ports. A water container is fluidly connected to the injection ports. An effluent collection container is fluidly connected to the first discharge port and the second discharge port. The inner housing defines a core space and an annular volume between the inner housing and the outer housing and is configured to enclose a core sample of a subterraneous formation in the core space. The injection ports are arranged in a longitudinal direction of the core space.

### CROSS-REFERENCE TO RELATED APPLICATION

The present disclosure claims the benefit of Saudi Patent Application No. 1020246327, filed on Nov. 11, 2024, with the Saudi Authority for Intellectual Property Office, which is incorporated herein by reference in its entirety.

### BACKGROUND

### Technical Field

The present disclosure is directed generally towards systems for oil reservoir flooding, and more particularly, directed towards a core flooding system and a method of acid diversion stimulation in the core flooding system.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

With the continued advancement of civilization, energy demands have increased exponentially. In order to meet the energy demands, fossil fuel has been used for decades. However, as commonly known, fossil fuel is a non-renewable energy resource and hence some of the fossil fuel reservoirs, alternatively referred to as oil wells, have aged and reached a stage where production costs outweigh economical profit margins. Further, a plurality of newly found oil wells have low initial productivity or yield due to various factors such as subterrarean morphology of oil wells. Hence, there is a requirement for efficient systems and methods to improve the yield from oil wells and improve productivity, resulting in enhanced oil extraction and low production costs.

Acid diversion stimulation (acidizing) has been used traditionally to improve the yield of new and old fossil fuel reservoirs. In general, the process of acidizing involves pumping an acid into a particular oil well in order to dissolve the rocks that line the contours of the oil well. Acidizing increases production rates by creating channels, called wormholes, into the rocks through which the oil or gas can flow into a reservoir. An additional benefit of acidizing the oil well is that it may help dissolve any loose debris found in the oil well. However, the use of an acid to stimulate a heterogeneous carbonate reservoir during matrix acidizing may lead to over-treating the high permeability zones, leaving low permeability zones untreated, which is particularly exacerbated in long horizontal sections. Therefore, the use of acid diverters for effective acid distribution across the heterogeneous carbonate reservoir is necessary. Conventionally, core flooding systems are utilized to acidize oil wells, where a single inlet line and a single outlet line are used or, in some cases, two outlet lines for dual-core flooding. In addition, matrix acidizing in a carbonate reservoir injects acid pressure below a fracture pressure to remove the formation damage and create wormholes. In rocks with varying properties, a significant difference in permeability may significantly decrease the effectiveness of stimulation treatments since the acid will predominantly flow into the zones with higher permeability. Inadequate design will result in unequal treatment of target areas and an unsuccessful treatment with acid. This phenomenon may be less beneficial in a thick or horizontal reservoir. Consequently, the oil and gas industry has extensively adopted mechanical and chemical diverters, selecting a different approach for each lithology to mitigate this effect. Hence, a better core flooding system is required that may provide an enhanced acid flow.

Accordingly, it is one object of the present disclosure to provide a core flooding system, that may circumvent the drawbacks, such as unequal acid distribution and low efficiency, of the methods and systems known in the art.

### SUMMARY

In an exemplary embodiment, a core flooding system is described. The core flooding system includes a core holder that includes an outer housing, an inner housing that is positioned inside the outer housing and sequentially includes a first end, a sidewall, and a second end. The core holder further includes a first discharge port positioned in the first end, a second discharge port positioned in the second end, and injection ports arranged along the sidewall. The core flooding system further includes an acid accumulator fluidly connected to the injection ports, a water container fluidly connected to the injection ports, and an effluent collection container fluidly connected to the first discharge port and the second discharge port. The inner housing defines a core space inside the inner housing and an annular volume between the inner housing and the outer housing, and the inner housing is configured to enclose a core sample of a subterraneous formation in the core space. The injection ports are arranged in a longitudinal direction of the core space and configured to deliver an injection liquid into the core space. The first discharge port and the second discharge port are configured to discharge an effluent from the core space.

In some embodiments, the core space is cylindrical, and the injection ports are arranged along the sidewall in a direction that is parallel to a central axis of the core space.

In some embodiments, the first discharge port and the second discharge port are arranged along the central axis of the core space.

In some embodiments, the injection ports are evenly spaced and form a straight line.

In some embodiments, the injection ports include five injection ports.

In some embodiments, the injection ports are each oriented perpendicular to the longitudinal direction of the core space and configured to deliver the injection liquid perpendicularly into the core space.

In some embodiments, the core flooding system further includes a fluid port extending through the outer housing and configured to deliver a confining liquid into the annular volume.

In some embodiments, the core flooding system further includes a pressure sensor positioned in the fluid port and configured to detect a pressure of the confining liquid.

In some embodiments, the sidewall of the inner housing does not have an opening that is fluidly connected to the annular volume.

In some embodiments, the core flooding system further includes a conduit that is positioned in the annular volume and fluidly connects the injection ports serially.

In some embodiments, the injection ports each extend through the outer housing, the annular volume and the sidewall of the inner housing.

In some embodiments, the core flooding system further includes a flexible sleeve positioned within the core holder and configured to confine the inner housing.

In some embodiments, the core flooding system further include pressure sensors positioned at the first end of the inner housing, the second end of the inner housing and the injection ports.

In some embodiments, the core flooding system further includes an oven, where the core holder and the acid accumulator are placed in the oven.

In some embodiments, the core flooding system further includes a CO₂ accumulator fluidly connected to the first end and the second end of the inner housing and one or more pumps configured to deliver deionized water from the water container to the injection ports and the annular volume and deliver the injection liquid from the acid accumulator to the injection ports.

In some embodiments, the core flooding system further includes a controller that is configured to inject deionized water perpendicularly into the subterraneous formation via the injection ports, inject the injection liquid perpendicularly into the subterraneous formation via the injection ports to form wormhole breakthroughs connected to the first discharge port, the second discharge port or both, inject again deionized water perpendicularly into the subterraneous formation via the injection ports, and analyze the wormhole breakthroughs in the subterraneous formation.

In another exemplary embodiment, a method of acid diversion stimulation in the core flooding system is described. The method includes injecting deionized water perpendicularly into the subterraneous formation via the injection ports, injecting the injection liquid perpendicularly into the subterraneous formation via the injection ports to form wormhole breakthroughs connected to the first discharge port, the second discharge port or both. The method further includes injecting again deionized water perpendicularly into the subterraneous formation via the injection ports, and analyzing the wormhole breakthroughs in the subterraneous formation.

In some embodiments, the core holder is placed so that the longitudinal direction of the core space is perpendicular to a gravity direction to simulate a horizontal borewell.

In some embodiments, the analyzing the wormhole breakthroughs includes executing computerized tomography.

In some embodiments, the wormhole breakthroughs include a first wormhole breakthrough connecting the first discharge port to a first injection port of the injection ports, a second wormhole breakthrough connecting the second discharge port to a second injection port of the injection ports. The first wormhole breakthrough and the second wormhole breakthrough are not directly connected with each other, and a branched hole branching from the second wormhole breakthrough towards the first discharge port, where the branched hole is longer than the second wormhole breakthrough, and the second discharge port has a higher permeability than the first discharge port.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A is a schematic diagram of a core flooding system for acid diversion stimulation of a subterraneous formation, according to certain embodiments;

FIG. 1B is a schematic diagram depicting an alternate configuration of the core flooding system of FIG. 1A, according to certain embodiments;

FIG. 2 is a flowchart depicting a method of acid diversion stimulation of the subterraneous formation in the core flooding system, according to certain embodiments;

FIG. 3 is a graph depicting pressure drop versus injected pore volumes in the subterraneous formation, according to certain embodiments;

FIG. 4 is a computerized tomography (CT) image depicting wormhole breakthroughs generated after acid diversion in a core sample of the subterraneous formation, according to certain embodiments;

FIG. 5 is an illustration of a non-limiting example of details of computing hardware used in a computing system corresponding to a controller of the core flooding system, according to certain embodiments;

FIG. 6 is an exemplary schematic diagram of a data processing system used within the computing system, according to certain embodiments;

FIG. 7 is an exemplary schematic diagram of a processor used with the computing system, according to certain embodiments; and

FIG. 8 is an illustration of a non-limiting example of distributed components which may share processing with the controller, according to certain embodiments.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

Aspects of this disclosure are directed towards a core flooding system and a method of acid diversion in the core flooding system. The present disclosure describes a core flooding system design with a plurality of acid injectors. Further, the present disclosure describes a method of acid diversion stimulation aimed at improved yield and productivity from horizontal oil wells, as well as, old and extracted heterogeneous carbonate reservoirs. Furthermore, the core flooding system is designed to be used in a plurality of subterranean geological formations without incurring hefty economical costs.

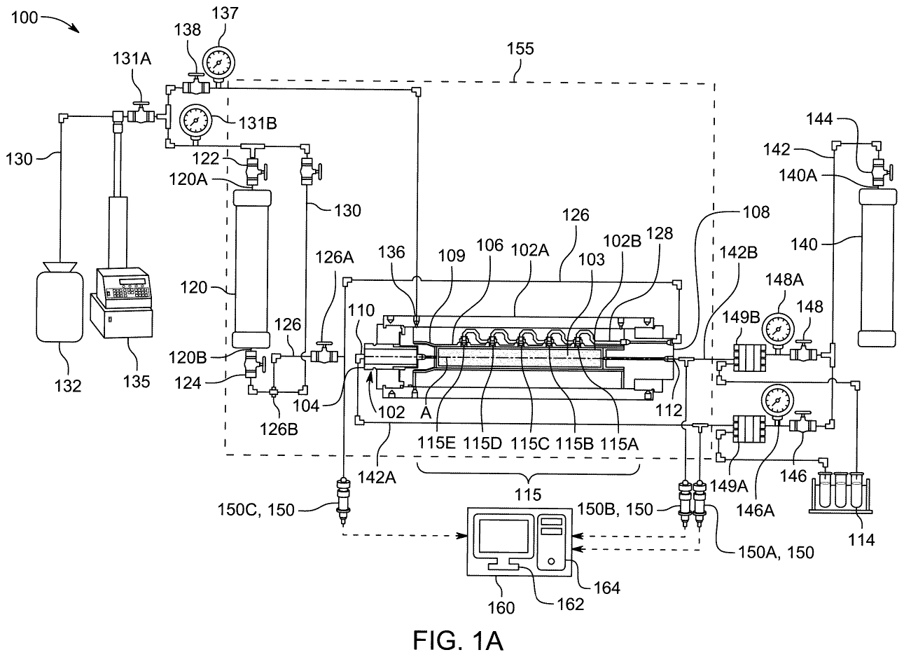

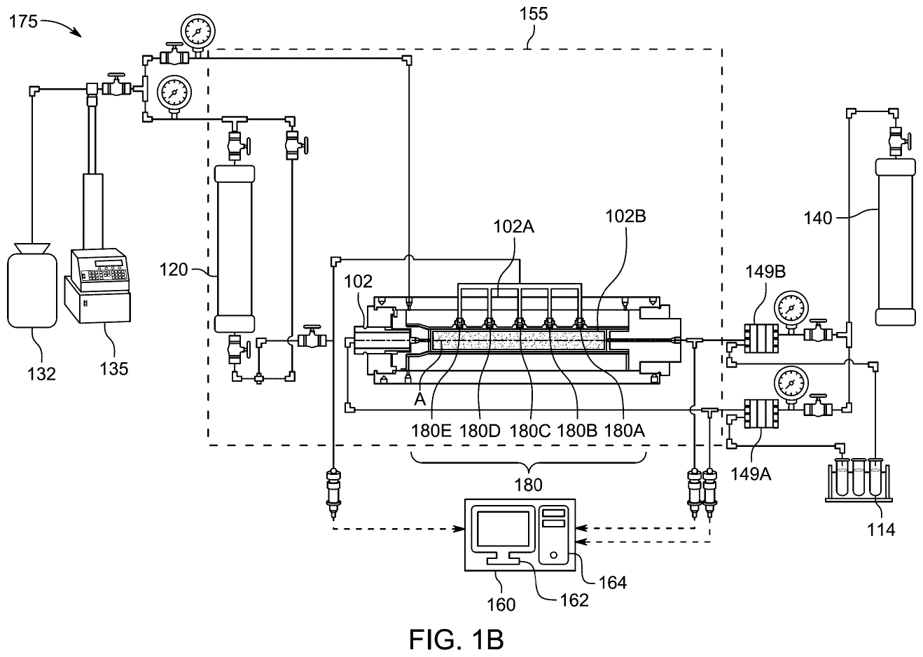

Referring to FIG. 1A, a schematic diagram of a core flooding system 100 is illustrated, according to certain embodiments. In general, core flooding systems are used in the oil and gas industry in order to flood an exhausted reservoir with an acid or acidic solution to extract remaining oil/gas present therein. The core flooding system 100 includes a core holder 102. In one implementation, the core holder 102 is cylindrical. The core holder 102 includes an outer housing 102A and an inner housing 102B. The inner housing 102B is positioned inside the outer housing 102A. In addition, the inner housing 102B defines a core space 103 inside the inner housing 102B and an annular volume between the inner housing 102B and the outer housing 102A. In general, an annular volume describes a total volume between an outer casing and a hole wall in oil and drilling applications, sometimes referred to as casing capacity.

Furthermore, the inner housing 102B is configured to enclose the core sample of the subterraneous formation in the core space 103. In one embodiment, the core space 103 is cylindrical in physical construction and is configured to enclose the core sample having a length of about 12 inches and a diameter of about 1.5 inches. The core sample has a similar cylindrical shape to mimic the core space 103, the outer and the inner housings 102A, 102B. In another embodiment, the core space 103 may enclose a larger or a smaller core sample, depending upon a site of employment of the core flooding system 100. Furthermore, the core holder 102 is placed so that a longitudinal direction of the core space 103 is perpendicular to a gravity direction to stimulate a horizontal borewell. In other words, the core holder 102 and the core space 103 are placed parallel to a ground and the gravitational pull is perpendicular to the direction of placement of the core holder 102. In some embodiments, the length of the core sample is 6-30 inches, preferably 8-21 inches, preferably 10-15 inches, preferably about 12 inches, and the diameter of the core sample is 0.5-3 inches, preferably 0.9-2.0 inches, preferably 1.2-1.5 inches, preferably about 1.5 inches. In some embodiments, the longitudinal direction of the core space 103 can have an angle of 60°-120°, preferably 70°-110°, preferably 80°-100°, preferably 85°-95°, preferably about 90° with the gravity direction. Of course it should be understood that dimensions are mentioned in this disclosure merely for illustrative purposes and are not limiting.

Moreover, the core flooding system 100 can operate and handle extreme conditions such as high acidity of pH of less than 4, preferably less than 3, preferably 1-2, high pressure of 5000-50000 psi, preferably 7000-30000 psi, preferably 9000-20000 psi, preferably about 10000 psi and a temperature ranging from 60° C. to 150° C., preferably 80° C. to 130° C., preferably 100° C. to 110° C. In order to withstand such demanding operating conditions, the inner housing 102B may be manufactured using corrosion resistance, high temperature and high pressure bearing materials such as, but not limited to, Hastelloy, which is nickel-super alloy specifically designed for oil and gas industry application to perform under high temperature, high pressure, and highly acidic conditions. In some embodiments, the inner housing 102B as well as the outer housing 102A may be manufactured using any other similar super-alloy(s) known in the art.

The inner housing 102B sequentially includes a first end 104, a sidewall 106, and a second end 108. As such, a left end of the core holder 102 is referred to as the first end 104 and a right end of the core holder 102 is referred to as the second end 108, while a circumferential wall, extracted in a longitudinal direction perpendicular to the first and the second ends 104, 108, is referred to as the sidewall 106. It may be noted that the sidewall 106 of the inner housing 102B does not have an opening that is fluidly connected to the annular volume, in order to provide an air-tight or fluid-tight enclosure for the core sample. In order to handle high pressure conditions and provide a structurally robust enclosure to the inner housing 102B, the core flooding system 100 includes a flexible sleeve 109 positioned within the core holder 102 and configured to confine the inner housing 102B. The flexible sleeve 109 may provide an adjustable, yet robust, enclosure to the inner housing 102B, in order to account for size changes that may occur in the core sample during a course of core flooding as carried out by the core flooding system 100. The flexible sleeve 109 further provides constant pressure for the core sample and keep the inner housing 102B confined to improve the yield for the core flooding system 100.

Further, the core holder 102 includes a set of discharge ports, as such, a first discharge port 110 positioned in the first end 104 and a second discharge port 112 positioned in the second end 108. The first discharge port 110 and the second discharge port 112 are configured to discharge an effluent, from the core space 103, that may be injected into the core sample enclosed in the core space 103. In one embodiment, the first discharge port 110 and the second discharge port 112 are arranged along a central axis ‘A’ of the core space 103. As such, the first and the second discharge ports 110, 112 are strategically placed at a center of the inner housing 102B, at both the first and second ends 104, 108. In some embodiments, the core flooding system 100 includes an effluent collection container 114. As such, the effluent collection container 114 is fluidly connected to the first discharge port 110 and the second discharge port 112, where the effluent collection container 114 is configured to store an effluent generated during a core flooding operation carried out by the core flooding system 100. In general, the effluent can include organic components, inorganic components, suspended solids, dissolved solids, and acids, naturally present or added during core flooding processes. The core flooding system 100 as described herein produces an effluent which may be high in acidic content, hence the effluent collection container 114 may be made of low-density polyethylene or any other polymer known in the art that resists acid deterioration.

The core holder 102 includes injection ports 115 (e.g. as shown by 115A, 115B, 115C, 115D and 115E) arranged along the sidewall 106 of the core holder 102. In some embodiments, the injection ports are arranged in the longitudinal direction of the core space 103 and configured to deliver an injection liquid into the core space 103. For example, the injection ports 115 can be arranged along the sidewall 106 in a direction that is parallel to the central axis ‘A’ of the core space 103. Further, the injection liquid, herein refers to an acidic fluid, may include about 15 percent by weight (wt. %) hydrochloric acid (HCl), about 6 wt. % viscoelastic surfactants (VES), about 10 wt. % calcium chloride (CaCl₂), and about 1% CI (corrosion inhibitor). However, the injection liquid may include varying concentrations of the aforementioned components, and other components known in the art, to accommodate specific requirements of the site of employment of the core flooding system 100. For instance, the injection liquid can include 5-25 wt. %, preferably 10-20 wt. % of, preferably 12.5-17.5 wt. % of HCl, 2-10 wt. %, preferably 4-8 wt. % of VES, 5-15 wt. %, preferably 7.5-12.5 wt. % of CaCl₂, and 0.5-3 wt. % of, preferably 0.7-1.5 wt. % of CI.

Furthermore, the injection ports 115 can be evenly spaced and form as straight line, in order to provide an even and equal flow of the injection liquid to the core sample enclosed in the core space 103. The straight-line arrangement of the injection ports 115 may help ensure that the injection ports 115 are inserted at appropriate depth in the core sample as required for desired performance of the core flooding system 100. An uneven depth may result in uneven amounts of injection liquid being injected into the core sample, resulting in flawed and inefficient operation of the core flooding system 100. In order to further ensure proper injection liquid flow, the injection ports 115 are each oriented perpendicular to the longitudinal direction of the core space 103 and configured to deliver the injection liquid perpendicularly into the core space 103, and the core sample enclosed therein. The inner housing 102B can be rotated around the central axis ‘A’ so the injection ports 115 may or may not be oriented in the gravity direction. When the injection ports 115 are oriented in the gravity direction, the perpendicular arrangement allows for a gravity assisted flow of the injection liquid into the core sample enclosed in the core space 103. In one embodiment, the injection ports 115 include five injection ports 115A, 115B, 115C, 115D, and 115E. The five injection ports 115A, 115B, 115C, 115D, and 115E are evenly spaced and arranged in a straight line with even depth insertion in the core sample enclosed in the core space 103. In another embodiment, the injection ports 115 may include more or fewer than five (e.g. 2, 3, 6, 8, 10, 20, 50, 100 or any values therebetween) injection ports in order to achieve desired output from the core flooding system 100, depending upon the site of employment of the core flooding system 100.

The core flooding system 100 further includes an acid accumulator 120 fluidly connected to the injection ports 115. The acid accumulator 120 is configured to store a predetermined volume of the injection liquid, and may or may not store deionized water. In some embodiments, the acid accumulator 120 is corrosion resistant and manufactured using super-alloys in order to resist low pH levels of the injection liquid stored inside the acid accumulator 120. The acid accumulator 120 includes an input 120A and an output 120B, as such, the input 120A further includes an input valve 122 and the output 120B includes an output valve 124. In some embodiments, the input 120A is defined at a top end of the acid accumulator 120 and the output 120B is defined at a bottom end of the acid accumulator 120. However, position of the input 120A and the output 120B may be interchangeable depending upon a requirement of the core flooding system 100. In some embodiments, the acid accumulator 120 is fluidly connected to the injection ports 115 via an injection conduit 126, configured with an inlet control valve 126A to control a flow of the injection liquid into the injection ports 115 from the acid accumulator 120. The injection conduit 126 is manufactured using corrosion resistant alloys and the inlet control valve 126A is disposed after the output 120B, a junction point 126B, and before a conduit 128. The core flooding system 100 includes the conduit 128 that is positioned in the annular volume and fluidly connects the injection ports 115, serially. As such, the injection conduit 126 is fluidly communicated with the conduit 128 in order to transfer the injection liquid from the acid accumulator 120 to the injection ports 115. The conduit 128 is further configured to distribute, an amount of the injection liquid to each injection port 115A, 115B, 115C, 115D, and 115E, serially.

In some embodiments, the input 120A and the output 120B of the acid accumulator 120 are configured to be in fluid communication with a water conduit 130, extending from a water container 132 which is configured to be fluidly connected to the injection ports 115. The water conduit 130 includes a water control valve 131A and a pressure gauge 131B. The water control valve 131A is disposed after the water container 132 and control a flow of the deionized water to the core flooding system 100. In addition, the pressure gauge 131B displays a pressure of water in the core flooding system 100. The water container 132 is configured to store deionized water for core sample saturation. In an example, the core sample enclosed in the core space 103 is saturated with deionized water for 24 hours before acid diversion stimulation is carried out with the injection liquid. The core flooding system 100 is a high pressure system and requires pressurized flow of fluids in the core flooding system 100, hence a pump 135 may be included to facilitate desired operation of the core flooding system 100. In some embodiments, the core flooding system 100 includes one or more pumps 135 configured to deliver deionized water from the water container 132 to the injection ports 115 in order to saturate the core sample. Further, the one or more pumps 135 may be configured to deliver the injection liquid from the acid accumulator 120 to the injection ports 115. In some examples, a single pump or a plurality of pumps may be connected to the core flooding system 100 depending on a use case of the core flooding system 100. Further, the core flooding system 100 as described herein may include an ISCO pump which may be a peristaltic pump or a hydraulic pump, having an improved injection rate of about 120 standard cubic centimeters per minute (cc/min). The water container 132 and the one or more pumps 135 may be disposed in close proximity to each other to improve efficiency.

In addition, the deionized water from the water container 132 may be utilized to pressurize the annular volume as described above, between the outer housing 102A and the inner housing 102B. In particular, the flexible sleeve 109 is pressurized at a confining pressure of 2000-7000 psi, preferably 2500-5000 psi, preferably 3000-4000 psi, preferably about 3500 psi. As such, the core flooding system 100 includes a fluid port 136 extending through the outer housing 102A and configured to deliver a confining liquid into the annular volume. According to the present disclosure, the confining liquid may be deionized water, supplied to maintain the confining pressure of about 3500 psi. The confining pressure exerted on the flexible sleeve 109, and subsequently the inner housing 102B is designated to regulate fluid flow in the core flooding system 100. However, excessive pressure or low pressure may hinder desired output from the core flooding system 100, hence, the core flooding system 100 includes a pressure sensor 137 positioned in the fluid port 136 and configured to detect a pressure of the confining liquid. The pressure sensor 137 may be disposed after a confining liquid valve 138 that is positioned after the water container 132 and before the fluid port 136. Particularly, the pressure sensor 137 is positioned in-line near an outlet of the confining liquid valve 138. The pressure sensor 137 may also be positioned on an inner wall of the outer housing 102A.

In some implementations of the core flooding system 100, a back pressure of 1000-5000 psi, preferably 1500-3500 psi, preferably 1750-2500 psi, preferably about 2000 psi is required to generate appropriate flooding of the core sample present in the core space 103. Hence, the core flooding system 100 includes a carbon dioxide (CO₂) accumulator 140 fluidly connected to the first end 104 and the second end 108 of the inner housing 102B. In some embodiments, the CO₂ accumulator 140 is configured to deliver carbon dioxide gas into the core holder 102 via a gas conduit 142 to apply about 2000 psi of back pressure into the core holder 102. The back pressure can help prevent premature exit of the injection liquid or the di-ionized water from the core space 103. The premature exit of the aforementioned fluids may prevent the core sample from absolute saturation, followed by desired acid diversion. According to the present disclosure, the CO₂ accumulator 140 includes an outlet 140A configured with an outlet control valve 144. The outlet control valve 144 is designed to cut-off CO₂ supply from the CO₂ accumulator 140 to the core holder 102. Further, the gas conduit 142 is split or branched into a first sub-conduit 142A and a second sub-conduit 142B. Specifically, the first sub-conduit 142A is configured to fluidly couple with the first discharge port 110 and the second sub-conduit 142B is configured to fluidly couple with the second discharge port 112. The first and the second sub-conduits 142A, 142B include a first flow control valve 146 and a second flow control valve 148, respectively. The first and the second flow control valves 146, 148 are configured to control a flow and a volume of the CO₂ being delivered to the first and the second discharge ports 110, 112 from the CO₂ accumulator 140. Furthermore, the first sub-conduit 142A includes a first pressure gauge 146A and the second sub-conduit 142B includes a second pressure gauge 148A. The first pressure gauge 146A is disposed in-line with the first sub-conduit 142A and before the first discharge port 110, the second pressure gauge 148A is disposed in-line with the second sub-conduit 142B and before the second discharge port 112. The first and the second pressure gauges 146A, 148A are configured to display a pressure amplitude of the incoming CO₂ from the CO₂ accumulator 140. In some embodiments, the CO₂ accumulator 140 is a pressurized cylinder, with enough gas capacity to generate the aforementioned back pressure. Further, the first sub-conduit 142A and the second sub-conduit 142B include a first distributor 149A and a second distributor 149B, respectively. The first and the second distributors 149A, 149B provide separation functionality to the core flooding system 100. In particular, the first and the second distributors 149A, 149B govern when to respectively allow flow of the CO₂ into the first and the second discharge ports 110, 112; and when to respectively allow effluent discharge from the first and the second discharge ports 110, 112 into the effluent collection container 114.

In order for monitoring of and control over multiple operations of the core flooding system 100, pressure sensors are employed at certain points to measure a pressure differential. In some embodiments, the core flooding system 100 includes pressure sensors 150 positioned at the first end 104 of the inner housing 102B, the second end 108 of the inner housing 102B and the injection ports 115. In particular, a first pressure sensor 150A is communicably coupled with the first end 104 of the inner housing 102B, a second pressure sensor 150B is communicably coupled with the second end 108 of the inner housing 102B, a third pressure sensor 150C is communicably coupled with the injection ports 115. In an example, the first and the second pressure sensors 150A, 150B are configured to measure a pressure of the effluent being discharged from the core holder 102 while the third pressure sensor 150C is configured to measure injection pressure of the injection liquid at the injection ports 115. In conjunction with each other, the pressure sensors 150 describe a pressure differential between input and output pressures in the core flooding system 100. The pressure differentials allow for a more accurate and efficient operation of the core flooding system 100.

As described in FIG. 1A, the core flooding system 100 includes an oven 155. In some embodiments, the core holder 102 and the acid accumulator 120 are placed in the oven 155. As described above, the core flooding system 100 may require high temperature conditions including a temperature of 60° C. to 150° C., preferably 80° C. to 130° C., preferably 100° C. to 110° C. for desired operational output, hence, the oven 155 provides the core flooding system 100 with an adjustable temperature gradient. The acid accumulator 120 is placed inside the oven 155 in order to increase a temperature of the injection liquid stored in the acid accumulator 120, further improving efficiency of the injection liquid in core flooding. In an example, the oven 155 may be heated to about 60° C. for an initiation of the flooding operation, at a confining pressure of 2000-7000 psi, preferably 2500-5000 psi, preferably 3000-4000 psi, preferably 3500 psi exerted by the confining liquid. Subsequently, the core sample present in the core holder 102 is heated to about 60° C. for 1-24 hours, preferably 2-12 hours, preferably 3-6 hours, preferably about 3 hours for even temperature distribution across all components of the core flooding system 100 that are enclosed in the oven 155. The oven 155 may be a large convectional style oven which is designed for elongated periods of operation. In some embodiments, the oven 155 may be operated using renewable energy resources in order to reduce overall cost associated with the core flooding system 100. In some implementations, the core flooding system 100 may include a drying oven (not shown), that may be used to dry offloaded core samples for elongated periods of about 24 hours at around 100° C. However, the drying step may not be required in certain implementations of the core flooding system 100.

In addition, the core flooding system 100 includes a controller 160. The core flooding system 100 is designed to have remote operation capabilities, hence, the controller 160 provides remote operation functionality to the core flooding system 100. Further, the controller 160 includes a display 162, and a processor 164 enclosing a memory. The memory including program instructions, which when executed by the processor 164 realizes the operation of the core flooding system 100. The controller 160 is configured to receive an input from a user of the core flooding system 100, where the input may be sent to the memory and include injection and analyze commands.

Referring to FIG. 1B, a schematic diagram of an alternate configuration of the core flooding system 100 is illustrated, according to certain embodiments. In particular, FIG. 1B describes a core flooding system 175. The embodiment of the core flooding system 175 herein is similar to the embodiment of the core flooding system 100 in FIG. 1A. Note that similar or identical components are labeled with similar or identical numerals unless specified otherwise. Descriptions have been provided above and will be omitted for simplicity purposes. The core flooding system 175 includes injection ports 180 and the core holder 102. Further, the integration of the injection ports 180 with the core holder 102, the circulation of injection liquid, and the flow of the deionized water and CO₂ within the core flooding system 175 closely resemble those in the core flooding system 100. The injection ports 180 include a first injection port 180A, a second injection port 180B, a third injection port 180C, a fourth injection port 180D, and a fifth injection port 180E, which are respectively similar to the injection ports 115A, 115B, 115C, 115D, and 115E of the core flooding system 100. Furthermore, the core flooding system 175, similar to the core flooding system 100, maintains fluid connection of the injection liquid in a closed loop and employs the one or more pumps 135 for a pressurized flow in the core flooding system 175. Also, the water container 132 is tasked with delivering deionized water to the core flooding system 175, and the CO₂ accumulator 140 is tasked with providing a carbon dioxide back pressure to the core flooding system 175. However, as illustrated in FIG. 1B, the core flooding system 175 introduces the alternate configuration in comparison to the core flooding system 100. The alternate configuration of the core flooding system 175, which is detailed in the subsequent paragraph(s), cater to specific operational requirements, thereby extending the versatility and applicability of the core flooding system 175.

The core flooding system 175, as illustrated in FIG. 1B, includes the injection ports 180 that extend outside the core holder 102. In some embodiments, the injection ports 180 each extend through the outer housing 102A, the annular volume and the sidewall 106 of the inner housing 102B. More specifically, the injection ports 180 are disposed inside the oven 155, and outside a perimeter of the outer housing 102A. Further, the injection ports 180 are lengthened to suit a requirement of the core flooding system 175, as such, the injection ports 180 extend to penetrate through, the outer housing 102A, the annular volume, the flexible sleeve 109, and the inner housing 102B. The injection ports 180 provide specific operational advancements by virtue of individual connectivity. In other words, the injection ports 180 does not have a serial connection like the injection ports 115, but a parallel configuration where each injection port from the injection ports 180 may be individually controlled. The individual control provides better adjustments for the injection liquid flow into the core space 103, and the injection liquid flow may be individually tweaked to better suit the requirements of the core flooding system 175. In an example, if the core flooding operation is suitably advanced in a first portion of the core sample, then the injection liquid flow may be increased in certain injection ports 180 and subsequently deliver more injection liquid by volume to a second portion of the core sample, where the core flooding operation may be lacking. In another example, such individual control can ensure that the flow rate and pressure of the injection liquid are the same for each of the injection ports 180. Similarly, the aforementioned operational adjustments may be incorporated vice-versa. In some embodiments, the controller 160 may be used to perform and fine-tune the aforementioned operational adjustments.

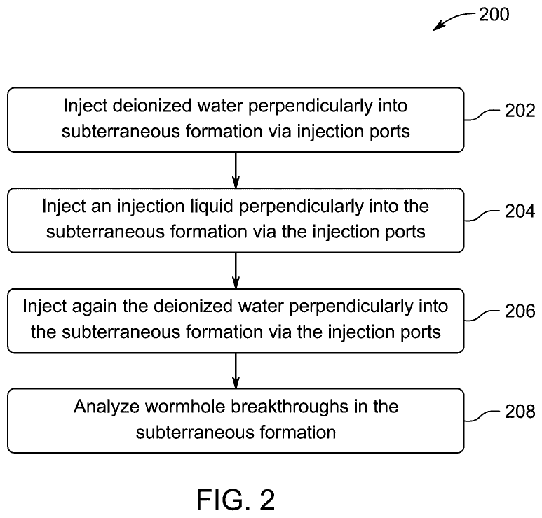

Referring to FIG. 2, a flowchart depicting a method 200 of acid diversion stimulation in the core flooding system 100 is illustrated, according to certain embodiments. The order in which the method 200 is described is not intended to be construed as a limitation, and any number of the described method steps can be combined to implement the method 200. Additionally, individual steps may be removed or skipped from the method 200 without departing from the spirit and scope of the present disclosure.

At step 202, the method 200 includes injecting deionized water perpendicularly into the subterraneous formation via the injection ports 115. In some embodiments, the core sample enclosed in the core space 103 of the core holder 102, is derived from the aforementioned subterraneous formation. In general, subterraneous formation may refer to any heterogeneous carbonated reservoir of oil, gas, or both. The deionized water is configured to perform a pre-flush of the core sample present in the core flooding system 100. In an exemplary core flooding operation, initially, a porosity of the core sample is measured using a helium porosimeter. Further, the core sample can undergo a preparation process which includes vacuuming the inner housing 102B of the core holder 102, in order to remove trapped air molecules in the core sample. The vacuum process may be carried out for about 3 hours. Furthermore, as mentioned in step 202 of the method 200, the core sample is injected with deionized water for about 24 hours to reach a saturation point. Moreover, after saturation, the core sample is subjected to the confining pressure of 3500 psi and a heat from the oven 155. The heating is done for about 3 hours for uniform temperature distribution. In addition, after reaching an equilibrium point, the effluent discharge pressures and injection pressure are recorded using the first, the second, and the third pressure sensors 150A, 150B, and 150C, respectively.

At step 204, the method 200 includes injecting the injection liquid perpendicularly into the subterraneous formation via the injection ports 115 to form wormhole breakthroughs connected to the first discharge port 110, the second discharge port 112, or both. In general, wormhole breakthroughs refer to high porosity channels generated within the core sample due to acid injection. According to the present disclosure, the wormhole breakthroughs include a first wormhole breakthrough connecting the first discharge port 110 to a first injection port 115A of the injection ports 115. As such, the injection liquid injected from the injection ports 115 travels through the subterraneous formation and exits at the first discharge port 110, thereby generating the first wormhole breakthrough. Further, the wormhole breakthroughs include a second wormhole breakthrough connecting the second discharge port 112 to a second injection port 115B of the injection ports 115, where the first wormhole breakthrough and the second wormhole breakthrough are not directly connected to each other. As such, the injection liquid injected from the second injection port 115B travels through the subterraneous formation and exits at the second discharge port 112, thereby creating the second wormhole breakthrough without hindering with the formation of the first wormhole breakthrough. In some embodiments, the wormhole breakthroughs further include a branched hole branching from the second wormhole breakthrough towards to first discharge port 110, where the branched hole is longer than the second wormhole breakthrough and the second discharge port 112 has a higher permeability than the first discharge port 110. As such, the branched hole is generated by the acid diversion stimulation, and the branched hole extends inside the core sample in multiple directions. Furthermore, the subterraneous formation may include more than the above specified amount of wormhole breakthroughs and branched hole. Further details pertaining to the permeability of the discharge ports 110, 112 are explained in subsequent paragraph(s) with the help of exemplary drawings.

As discussed earlier, the injection liquid can include 5-25 wt. %, preferably 10-20 wt. % of, preferably 12.5-17.5 wt. % of HCl, 2-10 wt. %, preferably 4-8 wt. % of VES, 5-15 wt. %, preferably 7.5-12.5 wt. % of CaCl₂, and 0.5-3 wt. % of, preferably 0.7-1.5 wt. % of CI. In a non-limiting example, the injection liquid includes 15 wt. % HCl, 10 wt. % CaCl₂, 6 vol. % VES, and 1% CI, and provides acid diversion stimulation in the core sample of the core holder 102. Acid diversion stimulation generates additional high porosity pathways or wormhole breakthroughs in the core sample by breaking down and dissolving organic and inorganic components of the core sample. The wormhole breakthroughs generated herein provides enhanced yield from a particular oil and gas reservoir, thereby improving profit fraction of an oil drilling operation. As described at step 204, the injection liquid is inserted perpendicularly to the direction of core holder 102 in order to take support of gravitational force, improving overall core flooding operation.

At step 206, the method 200 includes injecting again, the deionized water perpendicularly into the subterraneous formation via the injection ports 115. The core flooding system 100 includes post operation flushing mechanism. In an example, at step 206, the water container 132 and the pump 135 are prompted by the controller 160 to initiate the post operation flush. As such, the water container 132 releases a pre-determined quantity of deionized water and the pump 135 pressurizes the deionized water. Subsequently, the deionized water is delivered to the injection ports 115 via the water conduit 130, and injected perpendicularly into the core sample of the subterraneous formation. Furthermore, the core sample is offloaded to a drying oven (not shown) to further dry for 24 hours at about 100° C. Step 206 of the method 200 ensures that the core sample of the subterraneous formation is substantially acid-free and the wormhole breakthroughs are defined appropriately for further analysis and processing.

At step 208, the method 200 includes analyzing the wormhole breakthroughs in the subterraneous formation. In some embodiments, the analyzing of the wormhole breakthroughs includes executing computerized tomography (CT) or CT scans. In an example, the CT scan results of an exemplary core sample of Indiana limestone named “IL3” are listed in Table 1. The CT scan results demonstrate an efficacy of the core flooding system 100. Details of the wormhole breakthroughs generated through the core sample of the subterraneous formation are also shown in Table 1. As can be seen from Table 1, diversion efficiency is improved when chemical diverters such as VES are used. The results emphasize the significance of utilizing modern CT scans to assess acid diversion stimulation precisely, resulting in improved reservoir stimulation techniques and hydrocarbon extraction.

TABLE 1
CT scan results of generated wormhole breakthroughs

| | Wormhole | Sample | | Wormhole | Wormhole |
|---|---|---|---|---|---|
| Sample | volume | length | BV | volume | diameter |
| No | fraction | (mm) | (mm³) | (mm³) | (mm) |
| IL3 | 0.034 | 305.24 | 340.73 | 11.65 | 0.106 |

In an example, the Indiana limestone core (IL3) measuring about 1.5 inches in diameter and 12 inches in length was used for performing exemplary experiment using the aforementioned method steps 202, 204, and 206. A permeability of the IL3 sample was about 16 millidarcy (mD). Table 2 lists exemplary details of the acid diversion stimulation of the IL3 sample. As described in the present disclosure and the method 200, the exemplary experiment included the pre-flush with deionized water, followed by acid diversion with the injection liquid, and post-flush with the deionized water. It was observed that after injecting the injection liquid into the IL3 sample, the wormhole breakthrough took place from the second discharge port 112 after 10 minutes from the commencement of the experiment. Further, the wormhole breakthrough took place from the first discharge port 110 after 115 minutes from the commencement of the experiment. Hence, it may be understood from the above observations that the second discharge port 112 has higher permeability than the first discharge port 110. Furthermore, the injection liquid was continuously injected into the IL3 sample via the injection ports 115 even after the first and the second wormhole breakthroughs were generated.

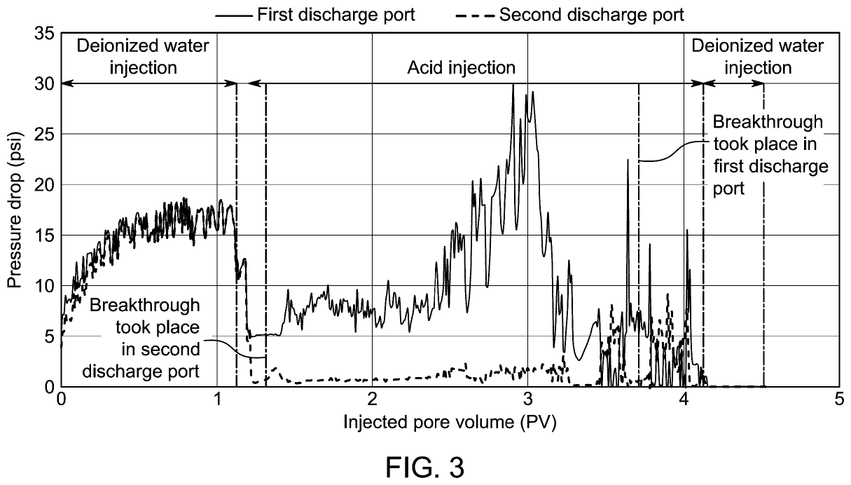

Referring to FIG. 3, a graph depicting pressure drop across the IL3 sample during the acid diversion stimulation is illustrated, according to certain embodiments. As can be seen from FIG. 3, the wormhole breakthroughs occurred at the second discharge port 112, with a lower pressure difference than the first discharge port 110. After that, the flow stopped at the second discharge port 112 due to an increased viscosity of the spent injection liquid, which led to temporary blockage, and the acid diverted to the lower permeability side, at the first discharge port 110. Further, FIG. 3 demonstrates the pressure drop versus the number of pore volumes (PV) of injection liquid injected into the IL3 sample. The wormhole breakthrough occurred at the second discharge port 112 after pumping 1.2 PV of the injection liquid. While, at the first discharge port 110, 3.4 PV of the injection liquid was consumed until the first wormhole breakthrough happened. In some cases, after the wormhole breakthroughs in the first and the second discharge ports 110, 112, the flow alternated between the first and second outlet points due to increased viscosity, which caused temporary flow blockage.

TABLE 2
Exemplary experimental details

| | Core | | | Perme- | |
|---|---|---|---|---|---|
| Core | dimensions | Inj. rate | | ability | Porosity |
| No. | (inches) | (cc/min) | Injection liquid | (mD) | (%) |
| IL3 | 1.5 × 12 | 1 | 15 wt. % HCl + | 16 | 16.61 |
| | | | 6 wt. % VES + | | |
| | | | 10 wt. % | | |
| | | | CaCl₂ + 1% CI | | |

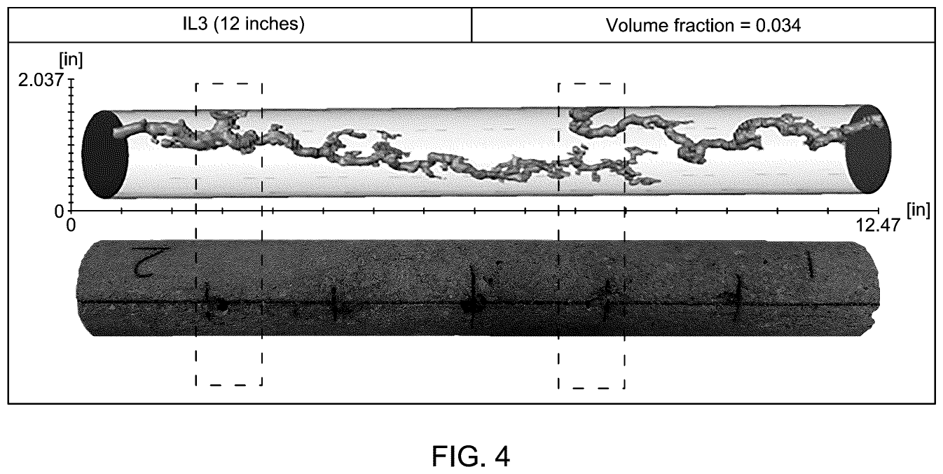

Further, referring to FIG. 4, an exemplary CT scan image of the IL3 sample post acid diversion stimulation is illustrated, according to certain embodiments. As can be seen from FIG. 4, the wormhole breakthroughs generated by the core flooding system 100 are in-line with the method 200 of acid diversion stimulation described with respect to FIG. 2.

Aspects of the present disclosure describe the core flooding systems 100, 175 in conjunction with the method 200. The injection ports 115, 180 used herein can improve reservoir yield by using the core flooding system 100, 175. Multiple injection points, as realized by the injection ports 115, 180, provide excellent wormhole breakthroughs and multiple perforations in the horizontally placed core sample. Further, the temperature and pressure conditions as described herein provide desirable conditions for improved efficiency of the core flooding systems 100, 175. Further, design and placement of the injection ports 115, 180 is simple and may be employed throughout the oil and gas industry, creating flexible use case scenarios for the core flooding systems 100, 175. Furthermore, CT scan results of the exemplary core flooding experiment provided valuable insights into the process improvements provided by the core flooding systems 100, 175 over the conventional flooding systems known in the art.

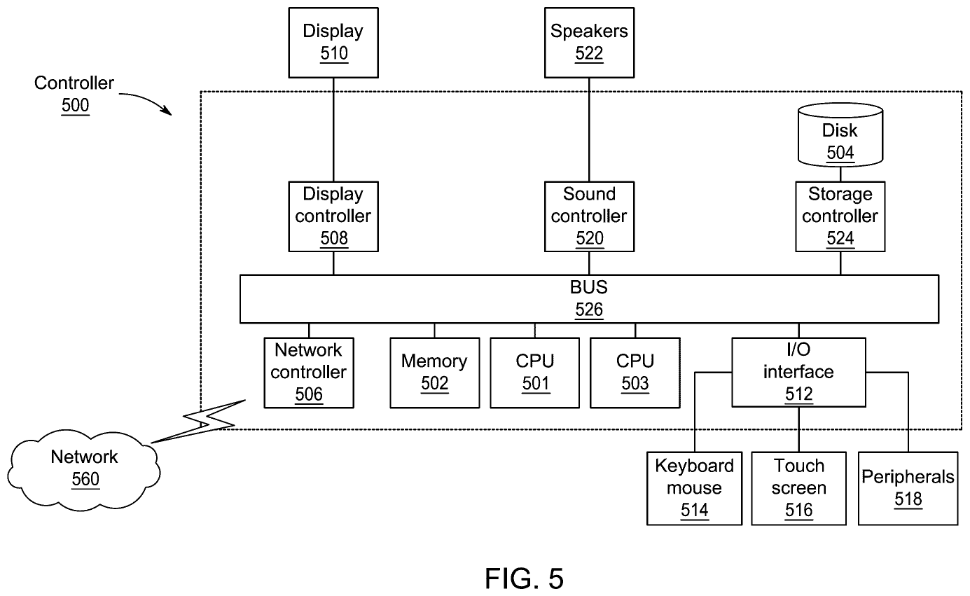

Next, further details of the hardware description of the computing environment according to exemplary embodiments is described with reference to FIG. 5. In FIG. 5, a controller 500 is described which is representative of the controller 160 of FIG. 1A and FIG. 1B, in which the controller is a computing device which includes a CPU 501 which performs the processes described above/below. The process data and instructions may be stored in memory 502. These processes and instructions may also be stored on a storage medium disk 504 such as a hard drive (HDD) or portable storage medium or may be stored remotely.

Further, the claims are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the computing device communicates, such as a server or computer.

Further, the claims may be provided as a utility application, background daemon, or component of an operating system, or combination thereof, executing in conjunction with CPU 701, 703 and an operating system such as Microsoft Windows 7, Microsoft Windows 10, Microsoft Windows 11, UNIX, Solaris, LINUX, Apple MAC-OS and other systems known to those skilled in the art.

The hardware elements in order to achieve the computing device may be realized by various circuitry elements, known to those skilled in the art. For example, CPU 501 or CPU 503 may be a Xenon or Core processor from Intel of America or an Opteron processor from AMD of America, or may be other processor types that would be recognized by one of ordinary skill in the art. Alternatively, the CPU 501, 503 may be implemented on an FPGA, ASIC, PLD or using discrete logic circuits, as one of ordinary skill in the art would recognize. Further, CPU 501, 503 may be implemented as multiple processors cooperatively working in parallel to perform the instructions of the inventive processes described above.

The computing device in FIG. 5 also includes a network controller 506, such as an Intel Ethernet PRO network interface card from Intel Corporation of America, for interfacing with network 560. As can be appreciated, the network 560 can be a public network, such as the Internet, or a private network such as an LAN or WAN network, or any combination thereof and can also include PSTN or ISDN sub-networks. The network 560 can also be wired, such as an Ethernet network, or can be wireless such as a cellular network including EDGE, 3G, 4G and 5G wireless cellular systems. The wireless network can also be Wi-Fi, Bluetooth, or any other wireless form of communication that is known.

The computing device further includes a display controller 508, such as a NVIDIA GeForce GTX or Quadro graphics adaptor from NVIDIA Corporation of America for interfacing with display 510, such as a Hewlett Packard HPL2445w LCD monitor. A general purpose I/O interface 512 interfaces with a keyboard and/or mouse 514 as well as a touch screen panel 516 on or separate from display 510. General purpose I/O interface also connects to a variety of peripherals 518 including printers and scanners, such as an OfficeJet or DeskJet from Hewlett Packard.

A sound controller 520 is also provided in the computing device such as Sound Blaster X-Fi Titanium from Creative, to interface with speakers/microphone 522 thereby providing sounds and/or music.

The storage controller 524 connects the storage medium disk 504 with communication bus 526, which may be an ISA, EISA, VESA, PCI, or similar, for interconnecting all of the components of the computing device. A description of the general features and functionality of the display 510, keyboard and/or mouse 514, as well as the display controller 508, storage controller 524, network controller 506, sound controller 520, and general purpose I/O interface 512 is omitted herein for brevity as these features are known.

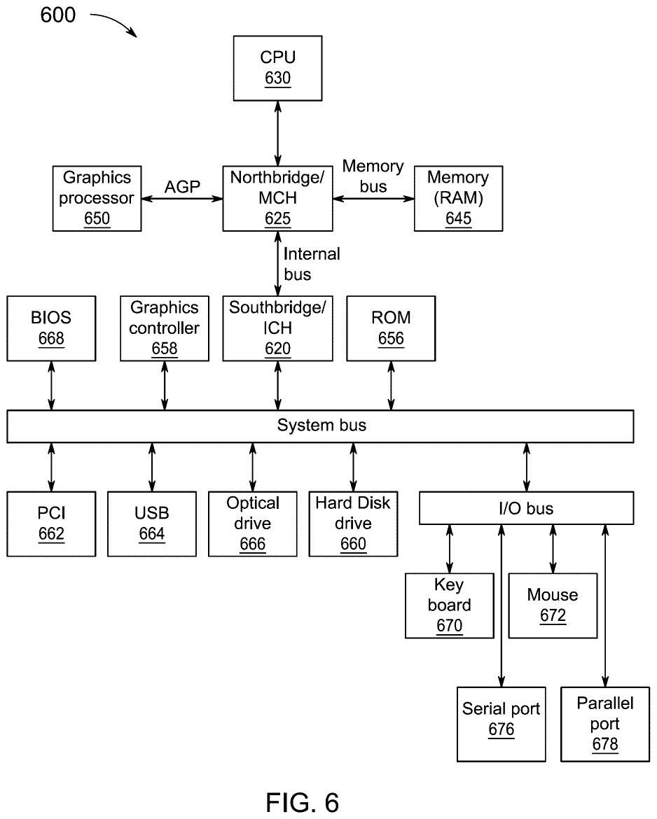

The exemplary circuit elements described in the context of the present disclosure may be replaced with other elements and structured differently than the examples provided herein. Moreover, circuitry configured to perform features described herein may be implemented in multiple circuit units (e.g., chips), or the features may be combined in circuitry on a single chipset, as shown on FIG. 6.

FIG. 6 shows a schematic diagram of a data processing system, according to certain embodiments, for performing the functions of the exemplary embodiments. The data processing system is an example of a computer in which code or instructions implementing the processes of the illustrative embodiments may be located.

In FIG. 6, data processing system 600 employs a hub architecture including a north bridge and memory controller hub (NB/MCH) 625 and a south bridge and input/output (I/O) controller hub (SB/ICH) 620. The central processing unit (CPU) 630 is connected to NB/MCH 625. The NB/MCH 625 also connects to the memory 645 via a memory bus, and connects to the graphics processor 650 via an accelerated graphics port (AGP). The NB/MCH 625 also connects to the SB/ICH 620 via an internal bus (e.g., a unified media interface or a direct media interface). The CPU Processing unit 630 may contain one or more processors and even may be implemented using one or more heterogeneous processor systems.

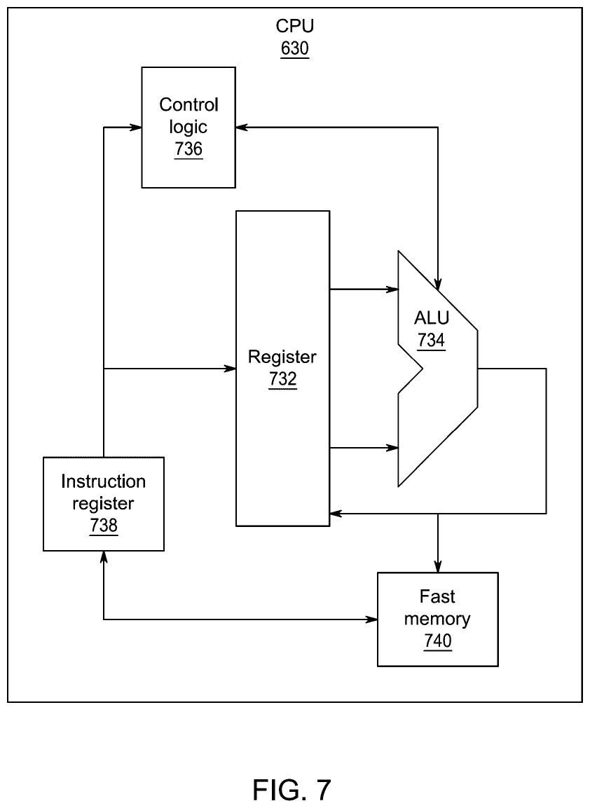

For example, FIG. 7 shows one implementation of CPU 630. In one implementation, the instruction register 738 retrieves instructions from the fast memory 740. At least part of these instructions are fetched from the instruction register 738 by the control logic 736 and interpreted according to the instruction set architecture of the CPU 730. Part of the instructions can also be directed to the register 732. In one implementation the instructions are decoded according to a hardwired method, and in another implementation the instructions are decoded according to a microprogram that translates instructions into sets of CPU configuration signals that are applied sequentially over multiple clock pulses. After fetching and decoding the instructions, the instructions are executed using the arithmetic logic unit (ALU) 734 that loads values from the register 732 and performs logical and mathematical operations on the loaded values according to the instructions. The results from these operations can be feedback into the register and/or stored in the fast memory 740. According to certain implementations, the instruction set architecture of the CPU 630 can use a reduced instruction set architecture, a complex instruction set architecture, a vector processor architecture, a very large instruction word architecture. Furthermore, the CPU 630 can be based on the Von Neuman model or the Harvard model. The CPU 630 can be a digital signal processor, an FPGA, an ASIC, a PLA, a PLD, or a CPLD. Further, the CPU 630 can be an x86 processor by Intel or by AMD; an ARM processor, a Power architecture processor by, e.g., IBM; a SPARC architecture processor by Sun Microsystems or by Oracle; or other known CPU architecture.

Referring again to FIG. 6, the data processing system 600 can include that the SB/ICH 620 is coupled through a system bus to an I/O Bus, a read only memory (ROM) 656, universal serial bus (USB) port 664, a flash binary input/output system (BIOS) 668, and a graphics controller 658. PCI/PCIe devices can also be coupled to SB/ICH 688 through a PCI bus 662.

The PCI devices may include, for example, Ethernet adapters, add-in cards, and PC cards for notebook computers. The Hard disk drive 660 and CD-ROM 666 can use, for example, an integrated drive electronics (IDE) or serial advanced technology attachment (SATA) interface. In one implementation the I/O bus can include a super I/O (SIO) device.

Further, the hard disk drive (HDD) 660 and CD-ROM 666 can also be coupled to the SB/ICH 620 through a system bus. In one implementation, a keyboard 670, a mouse 672, a parallel port 678, and a serial port 676 can be connected to the system bus through the I/O bus. Other peripherals and devices that can be connected to the SB/ICH 620 using a mass storage controller such as SATA or PATA, an Ethernet port, an ISA bus, a LPC bridge, SM-Bus, a DMA controller, and an Audio Codec.

Moreover, the present disclosure is not limited to the specific circuit elements described herein, nor is the present disclosure limited to the specific sizing and classification of these elements. For example, the skilled artisan will appreciate that the circuitry described herein may be adapted based on changes on battery sizing and chemistry or based on the requirements of the intended back-up load to be powered.

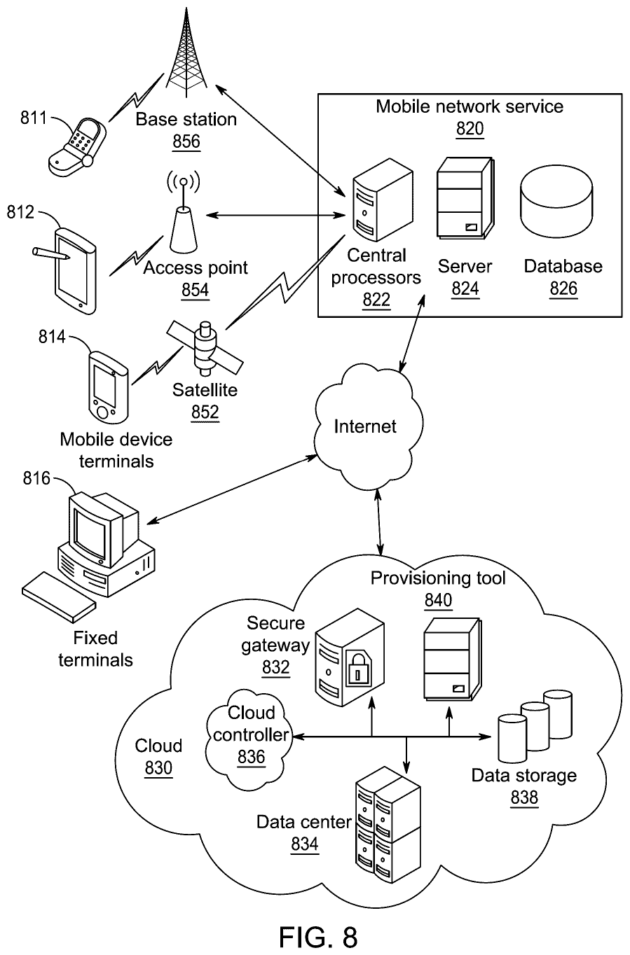

The functions and features described herein may also be executed by various distributed components of a system. For example, one or more processors may execute these system functions, wherein the processors are distributed across multiple components communicating in a network. The distributed components may include one or more client and server machines, such as cloud 830 including a cloud controller 836, a secure gateway 832, a data center 834, data storage 838 and a provisioning tool 840, and mobile network services 820 including central processors 822, a server 824 and a database 826, which may share processing, as shown by FIG. 8, in addition to various human interface and communication devices (e.g., display monitors 816, smart phones 810, tablets 812, personal digital assistants (PDAs) 814). The network may be a private network, such as a LAN, satellite 852 or WAN 854, or be a public network, may such as the Internet. Input to the system may be received via direct user input and received remotely either in real-time or as a batch process. Additionally, some implementations may be performed on modules or hardware not identical to those described. Accordingly, other implementations are within the scope that may be claimed.

The above-described hardware description is a non-limiting example of corresponding structure for performing the functionality described herein.

Numerous modifications and variations of the present disclosure are possible in light of the above teachings. It is therefore to be understood that within the scope of the appended claims, the invention may be practiced otherwise than as specifically described herein.

1. A core flooding system, comprising:
a core holder that comprises:
an outer housing,
an inner housing that is positioned inside the outer housing and sequentially includes a first end, a sidewall and a second end,
a first discharge port positioned in the first end,
a second discharge port positioned in the second end, and
injection ports arranged along the sidewall,
an acid accumulator fluidly connected to the injection ports,
a water container fluidly connected to the injection ports, and
an effluent collection container fluidly connected to the first discharge port and the second discharge port,
wherein the inner housing defines a core space inside the inner housing and an annular volume between the inner housing and the outer housing, and the inner housing is configured to enclose a core sample of a subterraneous formation in the core space,
wherein the injection ports are arranged in a longitudinal direction of the core space and configured to deliver an injection liquid into the core space,
wherein the first discharge port and the second discharge port are configured to discharge an effluent from the core space.

2. The core flooding system of claim 1, wherein:
the core space is cylindrical, and
the injection ports are arranged along the sidewall in a direction that is parallel to a central axis of the core space.

3. The core flooding system of claim 2, wherein:
the first discharge port and the second discharge port are arranged along the central axis of the core space.

4. The core flooding system of claim 3, wherein:
the injection ports are evenly spaced and form a straight line.

5. The core flooding system of claim 4, wherein:
the injection ports include five injection ports.

6. The core flooding system of claim 1, wherein:
the injection ports are each oriented perpendicular to the longitudinal direction of the core space and configured to deliver the injection liquid perpendicularly into the core space.

7. The core flooding system of claim 1, further comprising:
a fluid port extending through the outer housing and configured to deliver a confining liquid into an annular volume.

8. The core flooding system of claim 7, further comprising:
a pressure sensor positioned in the fluid port and configured to detect a pressure of the confining liquid.

9. The core flooding system of claim 7, wherein:
the sidewall of the inner housing does not have an opening that is fluidly connected to the annular volume.

10. The core flooding system of claim 1, further comprising:
a conduit that is positioned in the annular volume and fluidly connects the injection ports serially.

11. The core flooding system of claim 1, wherein:
the injection ports each extend through the outer housing, the annular volume and the sidewall of the inner housing.

12. The core flooding system of claim 1, further comprising:
a flexible sleeve positioned within the core holder and configured to confine the inner housing.

13. The core flooding system of claim 1, further comprising:
pressure sensors positioned at the first end of the inner housing, the second end of the inner housing and the injection ports.

14. The core flooding system of claim 1, further comprising:
an oven,
wherein the core holder and the acid accumulator are placed in the oven.

15. The core flooding system of claim 1, further comprising:
a CO2 accumulator fluidly connected to the first end and the second end of the inner housing; and
one or more pumps configured to deliver deionized water from the water container to the injection ports and the annular volume and deliver the injection liquid from the acid accumulator to the injection ports.

16. The core flooding system of claim 1, further comprising a controller that is configured to:
inject deionized water perpendicularly into the subterraneous formation via the injection ports;
inject the injection liquid perpendicularly into the subterraneous formation via the injection ports to form wormhole breakthroughs connected to the first discharge port, the second discharge port or both;
inject again deionized water perpendicularly into the subterraneous formation via the injection ports; and
analyze the wormhole breakthroughs in the subterraneous formation.

17. A method of acid diversion stimulation in the core flooding system of claim 1, comprising:
injecting deionized water perpendicularly into the subterraneous formation via the injection ports;
injecting the injection liquid perpendicularly into the subterraneous formation via the injection ports to form wormhole breakthroughs connected to the first discharge port, the second discharge port or both;
injecting again deionized water perpendicularly into the subterraneous formation via the injection ports; and
analyzing the wormhole breakthroughs in the subterraneous formation.

18. The method of claim 17, wherein:
the core holder is placed so that the longitudinal direction of the core space is perpendicular to a gravity direction to simulate a horizontal borewell.

19. The method of claim 17, wherein:
the analyzing the wormhole breakthroughs comprises executing computerized tomography.

20. The method of claim 17, wherein the wormhole breakthroughs comprise:
a first wormhole breakthrough connecting the first discharge port to a first injection port of the injection ports;
a second wormhole breakthrough connecting the second discharge port to a second injection port of the injection ports, wherein the first wormhole breakthrough and the second wormhole breakthrough are not directly connected with each other; and
a branched hole branching from the second wormhole breakthrough towards the first discharge port, wherein the branched hole is longer than the second wormhole breakthrough, and the second discharge port has a higher permeability than the first discharge port.
